(19) United States
(12) Patent Application Publication
(10) Pub. No.: US 2009/0220431 A1
(43) Pub. Date: Sep. 3, 2009

(54) MAGNETIC RESONANCE IMAGING CONTRAST AGENTS CONTAINING WATER-SOLUBLE NANOPARTICLES OF MANGANESE OXIDE OR MANGANESE METAL OXIDE

(76) Inventors: Jin-Woo Cheon, Seoul (KR); Young-Wook Jun, Gyeonggi-do (KR); Jae-Hyun Lee, Seoul (KR); Jung-Wook Suh, Gyeonggi-do (KR); Jin-Suck Suh, Seoul (KR); Seung-Jin Ko, Gyeonggi-do (KR); Yong-Min Huh, Seoul (KR); Ho-Taek Song, Seoul (KR)

Correspondence Address: DICKSTEIN SHAPIRO LLP, 1825 EYE STREET NW, Washington, DC, 20006-5403, US

(21) Appl. No.: 12/095,878

(22) PCT Filed: Dec. 1, 2006
(86) PCT No.: PCT/KR2006/005160

§ 371 Date: Nov. 10, 2008

(30) Foreign Application Priority Data
Dec. 2, 2005 (KR) 10-2005-0117038

(51) Int. Cl. A61K 49/06; A61P 43/00

(52) U.S. Cl. 424/9.32

(57) ABSTRACT

The present invention relates to a manganese-containing metal oxide nanoparticle-based magnetic resonance imaging (MRI) contrast agent, which is characterized in that: The core of it comprises 1 to 1000 nm-sized manganese-containing metal oxide nanoparticles which include MnO a (0<a<5) or MnMbOe (wherein M is at least one metal atom selected from the group consisting of a Group 1 or 2 element such as Li, Na, Be, Ca, Ge, Mg, Ba, Sr and Ra, a Group 13 element such as Ga and In, a transition metal element such as Y, Ta, V, Cr, Co, Fe, Ni, Cu, Zn, Ag, Cd and Hg, and lanthanide or actinide group elements such as La, Ce, Pr, Nd, Pm, Sm, Eu, Gd, Tb, Dy, Ho, Er, Tm and Yb, 0<b<5 and 0<c<10); preferably MnM′dFeeOf (wherein M′ is at least one metal atom selected from the group consisting of a Group 1 or 2 element such as Li, Na, Be, Ca, Ge, Mg, Ba, Sr and Ra, a Group 13 element such as Ga and In, a transition metal element such as Y, Ta, V, Cr, Co, Fe, Ni, Cu, Zn, Ag, Cd and Hg, and lanthanide or actinide group elements such as La, Ce, Pr, Nd, Pm, Sm, Eu, Gd, Tb, Dy, Ho, Er, Tm and Yb, 0<d<5, 0<e<5, and 0<f<15). In addition, the nanoparticles include water-soluble manganese-containing metal oxide nanoparticles which is characterized in that they are soluble in water themselves or stable in an aqueous media as being coated with a water-soluble ligand and they possess enhanced magnetic properties and MRI contrast effect. Also the water soluble manganese-containing metal oxide nanoparticles are coupled with an bioactive material such as chemical molecules or bio-functional molecules, and thus the nanoparticles can be used as an MRI contrast agent for target specificity and cell tracking.

### TECHNICAL FIELD

The present invention relates to a manganese-containing metal oxide nanoparticle-based magnetic resonance imaging (MRI) contrast agent, which is characterized in that: (1) The core of it comprises 1 to 1000 nm-sized manganese-containing metal oxide nanoparticles which include MnOₐ (0<a≦5) or MnM_{b}O_{c} (wherein M is at least one metal atom selected from the group consisting of a Group 1 or 2 element such as Li, Na, Be, Ca, Ge, Mg, Ba, Sr and Ra, a Group 13 element such as Ga and In, a transition metal element such as Y, Ta, V, Cr, Co, Fe, Ni, Cu, Zn, Ag, Cd and Hg, and lanthanide or actinide group elements such as La, Ce, Pr, Nd, Pm, Sm, Eu, Gd, Tb, Dy, Ho, Er, Tm and Yb, 0<b≦5 and 0<c≦10); preferably MnM′_{d}FeₑO_{f} (wherein M′ is at least one metal atom selected from the group consisting of a Group 1 or 2 element such as Li, Na, Be, Ca, Ge, Mg, Ba, Sr and Ra, a Group 13 element such as Ga and In, a transition metal element such as Y, Ta, V, Cr, Co, Fe, Ni, Cu, Zn, Ag, Cd and Hg, and lanthanide or actinide group elements such as La, Ce, Pr, Nd, Pm, Sm, Eu, Gd, Tb, Dy, Ho, Er, Tm and Yb, 0<d≦5, 0<e≦5, and 0<f≦15); and most preferably MnFe₂O₄; (2) The nanoparticles include water-soluble manganese-containing metal oxide nanoparticles which is characterized in that they are soluble in water themselves or stable in aqueous media as being coated with a water-soluble ligand and they possess enhanced magnetic properties; (3) This invention also provides hybrid nanostructures of above-mentioned manganese-containing metal oxide nanoparticles coupled with bioactive materials such as chemical molecules or bio-functional molecules; and (4) The present invention relates to development of a MR contrast agent by using the nanomaterials described in the above (1) to (3).

### BACKGROUND ART

Nanotechnology is a technique for controlling or manipulating materials at the atomic or molecular level, and is for fabricating new materials and devices. The nanotechnology has wide application, such as electronics, materials, communications, machines, medicals, agriculture, energy, and environments.

At present, nanotechnology is under development in various fields, which fall typically into three categories. First one relates to a technique for synthesizing new ultra-fine materials with nanoscale materials. Second one relates to a technique for preparing a device by combination or alignment of nanoscale materials, said device exhibiting a specific function. Third one relates to a technique, so-called “nano-bio,” for applying nanotechnology to biotechnology.

In the nano-bio field, magnetic nanoparticles are used in a wide variety of applications such as separation of biomaterials, diagnostic probes for magnetic resonance imaging, biosensors including giant magnetoresistance sensor, microfluidic sensors, drugs/genes delivery, and magnetic hyperthermia.

In particular, magnetic nanoparticles can be used as a diagnostic probe for MRI. Under an applied magnetic field, the magnetic nanoparticles are magnetized, which leads the shortening a spin-spin relaxation time of the protons in water molecules which surround the nanoparticles, thereby result in MR signal enhancement. Accordingly, such MR signal enhancement can be applied to disease diagnosis or observation of biological events at the molecular/cellular level.

U.S. Pat. No. 6,274,121, discloses superparamagnetic nanoparticles (e.g. iron oxide), to whose surfaces are bound inorganic substances having binding sites for coupling to tissue-specific binding substances, diagnostic or pharmacologically active substances.

U.S. Pat. No. 6,638,494, relating to paramagnetic nanoparticles comprising metals (e.g. iron oxide), discloses a method for preventing nanoparticles from aggregation and sedimentation under an applied magnetic field or gravity by means of carboxylic acids which coats the surface of the nanoparticles. As the specific carboxylic acid, an aliphatic dicarboxylic acid such as maleic acid, tartaric acid and glucaric acid; or an aliphatic polydicarboxylic acid such as citric acid, cyclohexane and tricarboxylic acid was used.

U.S. Pat. No. 5,746,999, relating to paramagnetic nanoparticles comprising metals (e.g. iron oxide), discloses nanoparticles which is coated with silica, attached with dextran and then applied in in vivo MRI.

U.S. Pat. Nos. 5,069,216 and 5,262,176 disclose a colloid including paramagnetic nanoparticles comprising metals (e.g. iron oxide), wherein the nanoparticles are solubilized by coating with a polysaccharide such as dextran, and they are used for MRI of an organ such as the liver and the stomach.

U.S. Patent Application Publication No. 2004/0058457 discloses functional nanoparticles coated with a monolayer of bifunctional peptide which can be conjugated with various biopolymers including DNA and RNA.

U.S. Pat. No. 5,336,506 discloses iron oxide magnetic nanoparticles coated with dextran to which folic acid is attached, and capable of selectively probing a cancer cell, wherein it is used for in vitro MRI diagnosis of a cancer cell.

U.S. Pat. No. 4,770,183 discloses magnetic iron oxide nanoparticles coated with dextran and a proteins (e.g. BSA), which is applied to the liver imaging of the human body and biodistribution by means of magnetic resonance imaging.

Korean Patent Application No. 10-1998-0705262 discloses particles comprising superparamagnetic iron oxide core particle coated with a starch and any polyalkylene oxide, and an MRI contrast agent comprising the same.

The magnetic nanoparticles used for these MRI contrast agents should fulfill the following requirements for their high performance MRI applications:

1) They should have high magnetic susceptibility enough to sensitively react in the magnetic field;

2) They should exhibit excellent MRI contrast effects;

3) They should be stably transferred and distributed in vivo, that is, in a water soluble environment;

4) They should easily bind with a biologically active material; and

5) They should exhibit low toxicity and high biocompatibility.

MRI performs excellent 3-dimensional tomography with high spatial resolution, but its low diagnostic sensitivity has been a major drawback. In order to solve the above problems, there is an urgent need of a development of magnetic nanoparticles having excellent magnetic properties and a contrast effect.

However, the conventional iron oxide-based nanoparticles including MRI contrast agents disclosed in the above-described patent publications or CLIO, Feridex, and Resovist, etc. hetherto known, have low magnetic susceptibility (60 to 90 emu/gFe), and thus, low MRI contrast effects (e.g., low R2 relaxivity coefficient (60 to 150 L□mol¹sec⁻¹)). They also exhibit a reduced signal enhancement as an MRI contrast agent, and thus it have been pointed out that they have significant problems in the magnetic resonance imaging diagnosis.

### DISCLOSURE OF INVENTION

### Technical Problem

The object of the present invention is to overcome the problems of the conventional iron oxide nanoparticles, and to provide water soluble manganese-containing metal oxide nanoparticles as a new-concept MRI contrast agent, which have an excellent magnetic properties and excellent MRI contrast effects, and which improves remarkably the magnetic resonance imaging diagnosis effect due to high stability in an aqueous solution.

### Technical Solution

The present inventors developed water soluble manganese-containing metal oxide nanoparticles having highly enhanced magnetic properties, good colloidal stability in aqueous media and biocompatibility, and being capable of easily binding with biologically functional components, instead of using the conventional iron oxide nanoparticles. Further, they developed hybrid nanoparticles of manganese-containing metal oxide nanoparticles conjugated with chemical or biological molecules such as proteins, antigens, antibodies, peptides, nucleic acids, and enzymes to the manganese-containing metal oxide nanoparticles via a linker ligand. These water soluble manganese-containing metal oxide nanoparticles, and manganese-containing metal oxide nanoparticles enables ultra-sensitive diagnosis of cancer with highly improved detection sensitivity, which allow diagnosis with high-sensitivity in the magnetic resonance imaging.

### ADVANTAGEOUS EFFECTS

The water soluble manganese-containing metal oxide nanoparticles, and water soluble manganese-containing metal oxide hybrid nanoparticles according to the present invention have uniform sizes, are stable particularly in an aqueous solution, and exhibit very excellent magnetic properties. They remarkably increase the magnetic properties, as compared with the conventional iron oxide nanoparticles, and thus show remarkably enhanced MRI sensitivity. The water soluble manganese-containing metal oxide nanoparticles or nano hybrid conjugated with the biomaterials thereof can be used in drastic improvement on the conventional MRI and in the diagnostic treatment system.

### BRIEF DESCRIPTION OF THE DRAWINGS

FIG. 1 illustrates comparison in the MRI contrast effects of manganese-containing metal oxide (in this case MnFe₂O₄) nanoparticles with manganese-free metal oxide nanoparticles including iron oxide (Fe₃O₄), cobalt ferrite (CoFe₂O₄), nickel ferrite (NiFe₂O₄) nanoparticles. All nanoparticles have identical size of ˜12 nm and are coated with 2,3-dimercaptosuccinic acid. FIG. 1(a) illustrates transmission electron microscope images of the obtained nanoparticles. FIG. 1(b) illustrates magnetization value at 1.5 T. FIGS. 1(c) and 1(d) illustrate the T2 spin-spin MRI's (c) of each nanoparticle from comparison of the T2 spin-spin relaxation MRI contrast effect of each nanoparticle, and the R2 (=1/T2) relaxivity coefficient, respectively. FIG. 1(e) illustrates comparison of the MRI contrast effects of the manganese ferrite nanoparticles coated with various ligands and the iron oxide nanoparticles, wherein (1) and (2) depict the manganese ferrite nanoparticles and the iron oxide nanoparticles, respectively, coated with dextran, (3) and (4) depict the manganese ferrite nanoparticles and the iron oxide nanoparticles, respectively, coated with 3-carboxypropylphosphate, (5) and (6) depict the T2 spin-spin relaxation MRI result of the aqueous solution containing the manganese ferrite nanoparticles and the iron oxide nanoparticles, respectively, coated with mercaptosuccinic acid. FIG. 1(f) illustrates the comparison of the R2 relaxivity co-efficient of the iron oxide nanoparticles and the manganese ferrite nanoparticles surrounded by the ligands having the same size.

FIG. 2 illustrates size-dependent MRI contrast effects of the manganese ferrite and iron oxide nanoparticles. FIG. 2(a) illustrates TEM images of 6 nm, 9 nm, and 12 nm-sized manganese ferrite nanoparticles, FIG. 2(b) illustrates hysteresis loops of the manganese ferrite nanoparticles in various sizes, FIG. 2(c) illustrates size-dependent T2 spin-spin relaxation MR images of the manganese ferrite nanoparticles, FIG. 2(d) illustrates size-dependent R2 relaxivity coefficient of the manganese ferrite and iron oxide nanoparticles.

FIG. 3 illustrates colloidal stability tests of the manganese ferrite nanoparticles coated with various ligands. FIG. 3(a) illustrates agarose gel electrophoretic pictures of the 6 nm, 9 nm, and 12 nm-sized, manganese ferrite nanoparticles coated with dimethyl mercapto succinic acid. FIGS. 3(b) to 3(i) illustrate a salt (NaCl) solution of the manganese ferrite nanoparticles coated with various ligands, and the test on the colloidal stability and the solubility thereof in accordance with the change in pH.

FIG. 4(a) illustrates the synthetic scheme of manganese ferrite (12 nm) nanoparticles-herceptin hybrids and the FIG. 4(b) illustrates the result of Coomassie Blue protein staining of the synthesized nano hybrid material on agarose gel electrophoresis.

FIG. 5 illustrates the evaluation on breast cancer MRI diagnostic sensitivity in vitro using the manganese ferrite nanoparticles-herceptin hybrid. FIG. 5(a) illustrates the relative HER2/neu expression levels in cell lines (Bx-PC-3, MDA-MB-231, MCF-7, and NIH3T6.7). FIG. 5(b) illustrates the T2-weighted MR images of cell lines treated with manganese ferrite nanoparticles-herceptin hybrid. FIG. 5(c) illustrates the T2-weighted MR images of cell lines treated with cross-linked iron oxide (CLIO) as control which is a per se known, representative molecule MRI contrast agent. FIG. 5(d) illustrates the plot of relative HER2/neu expression level for each cell lines versus R2 enhancement, from the result depicted in FIGS. 5(b) and 5(c).

FIG. 6 illustrates the result of the cytotoxicity test of the manganese ferrite nanoparticles and the manganese ferrite nanoparticles-herceptin hybrid. FIGS. 6(a) and 6(b) illustrate the cytotoxicity effects of manganese ferrite nanoparticles on two different cell lines, HeLa and HepG2, and FIGS. 6(c) and 6(d) illustrate the cytotoxicity effects of manganese ferrite nanoparticles-herceptin hybrids on two different cell lines, HeLa and HepG2.

FIG. 7(a) illustrates TEM image of the manganese-containing metal oxide nanoparticles, and FIG. 7(b) illustrates the T2 spin-spin relaxation MR images of the nanoparticles. As the control group, water without the nanoparticles was used.

FIG. 8 illustrates T1 spin-lattice MR images by the release of the manganese ions of the manganese-containing metal oxide nanoparticles. FIG. 8(a) illustrates T1-weighted MR images of the Mn²⁺ ion as a reference material, and FIGS. 8(b) and 8(c) illustrate the MR images showing the T1 spin-lattice contrast effect by the release of the manganese ions when the manganese ferrite nanoparticles and the manganese-containing metal oxide nanoparticles is dissolved in an aqueous solutions at pH 2, 4, and 7. FIGS. 8(d) and 8(e) illustrate the plot of the R1 (=1/T1) relaxation signals from the MR images of FIGS. 8(b) and 8(c).

FIG. 9 illustrates in vivo MR detection of small size (50 mg, 2 mm×5 mm×5 mm) breast cancer using manganese ferrite nanoparicle (12 nm)-herceptin hybrids. FIGS. 9(a) to 9(c) illustrate the color maps of T2 spin-spin relaxation MR images of a mouse implanted with the cancer cell line, at different time points after injection of manganese ferrite nanoparticles-herceptin hybrids (preinjection (a), 1 hour (b) and 2 hours (c) after injection), FIGS. 9(d) to 9(f) illustrate the MR images after injection of the iron oxide nonoparticle-herceptin hybrids under the same conditions to those of the manganese ferrite nanoparticles-herceptin hybrid, and FIGS. 9(g) to 9(i) illustrate the MR images after injection of the CLIO nonoparticle-herceptin hybrids. In these Figures, color gradually changes at tumor site, from red (that is, low R2) to blue (that is, high R2). FIG. 9(j) illustrates plot of R2 change (ΔR2/R2control) versus time of the breast cancer tissues in the images shown in FIGS. 9(a) to 9(i).

FIG. 10 illustrates the gamma camera images from a nude mouse having the breast cancer, at 2 hours after injection (a), and 24 hours after injection (b) of ¹¹¹In-labeled manganese ferrite nanoparticles-herceptin hybrid. FIG. 10(c) is a table illustrating a biodistribution (% ID/g: percent injection dose per gram of organ) of the manganese ferrite nano hybrids as measured with a gammacounter of the organs explanted from the nude mouse, which was sacrificed 24 hours after injection.

FIG. 11(a) is a scheme of the magnetic-optical dual mode nanoparticles, obtained by coupling fluorescein isocyanate (FITC) to manganese ferrite nanoparticles, FIG. 11(b) illustrates photoluminescence spectrum of fluorescent properties and the fluorescence image, and FIG. 11(c) illustrates the R2 spin-spin relaxivity coefficient and the MR image of the dual mode nanoparticles.

### BEST MODE FOR CARRYING OUT THE INVENTION

As used in the specification of the present invention, “manganese-containing metal oxide nanoparticles” means nanoparticles of manganese oxide or manganese metal oxide. In the specification of the present application, the nanoparticles of manganese oxide or manganese metal oxide or manganese metal oxide are commonly referred to as “manganese-containing metal oxide nanoparticles.”

As used in the specification of the present invention, the “manganese-containing metal oxide nanoparticles” means nano-scale particles having a diameter in the range of 1 nm to 1000 nm, preferably 2 nm to 100 nm, as well as a solubility in water of at least 1 □/ml and a hydrodynamic radius of 1000 nm or less.

As used in the present invention, the “water soluble manganese-containing metal oxide nanoparticles” means nanoparticles having a water soluble multi-functional group ligand bound to and surrounding the manganese-containing metal oxide nanoparticles, or being capable of being dissolved or dispersed themselves in an aqueous solution without binding to a specific ligand.

As used in the present invention, the “water soluble manganese-containing metal oxide hybrid nanoparticles” means materials having the water soluble manganese-containing metal oxide nanoparticles bound to the chemically functional materials (e.g., monomers, polymers, and inorganic supports) or biologically functional materials (e.g., cells, proteins, peptides, antigens, genes, antibodies and enzymes).

The water soluble manganese-containing metal oxide nanoparticles according to the present invention can be provided in a variety of forms, the forms will depend on which manganese-containing metal oxide and the multi-functional group ligand is selected.

The manganese-containing metal oxide of the present invention is MnOₐ (0<a≦5) or MnM_{b}O_{c} (wherein M is at least one metal atom selected from the group consisting of a Group 1 or 2 element such as Li, Na, Be, Ca, Ge, Mg, Ba, Sr and Ra, a Group 13 element such as Ga and In, a transition metal element such as Y, Ta, V, Cr, Co, Fe, Ni, Cu, Zn, Ag, Cd and Hg, and lanthanide or actinide group elements such as La, Ce, Pr, Nd, Pm, Sm, Eu, Gd, Tb, Dy, Ho, Er, Tm and Yb, 0<b≦5 and 0<c≦10); preferably MnM′_{d}FeₑO_{f} (wherein M′ is at least one metal atom selected from the group consisting of a Group 1 or 2 element such as Li, Na, Be, Ca, Ge, Mg, Ba, Sr and Ra, a Group 13 element such as Ga and In, a transition metal element such as Y, Ta, V, Cr, Co, Fe, Ni, Cu, Zn, Ag, Cd and Hg, and lanthanide or actinide group elements such as La, Ce, Pr, Nd, Pm, Sm, Eu, Gd, Tb, Dy, Ho, Er, Tm and Yb, 0<d≦5, 0<e≦5, and 0<f≦15); and most preferably MnFe₂O₄.

As used in specification of the present invention, the “water soluble multi-functional group ligand” can include (a) an adhesive region (LI), and can further include (b) a reactive region (LII), or (c) a crosslinking region (LIII). Hereinbelow, the water soluble multi-functional group ligand will be described in detail.

The “adhesive region (LI)” means a portion of a multi-functional group ligand, comprising a functional group capable of binding to the nanoparticles, and preferably an end portion thereof. Accordingly, it is preferable that the adhesive region comprises a functional group having high affinity with the materials constituting the nanoparticles. Here, the nanoparticles can be attached to the adhesive regions by an ionic bond, a covalent bond, a hydrogen bond, a hydrophobic bond, or a metal-ligand coordination bond. Thus, a variety of the adhesive region of the multi-functional group ligand can be selected depending on the materials constituting the nanoparticles. For example, the adhesive region using ionic bond, covalent bond, hydrogen bond, or metal-ligand coordination bond can comprise —COOH, —NH₂, —SH, —CONH₂, —PO₃H, —PO₄H, —SO₃H, —SO₄H, —N₃, —NR₃OH (R═CₙH₂ₙ₊₁, 0≦n≦16) or —OH, and the adhesive region using the hydrophobic bond can comprise a hydrocarbon chain containing 2 or more carbon atoms, but not limited thereto.

The “reactive region (LII)” means a portion of the multi-functional group ligand comprising a functional group capable of binding to the active ingredient, and preferably the other end portion opposite the adhesive region. The functional group of the reactive region can be varied depending on the kinds of the active ingredients and their chemical formulae (see Table 1). In the present invention, the reactive region can comprise —SH, —COOH, —NH₂, —OH, —PO₃H, —PO₄H₂, —SO₃H, —SO₄H—NR⁴⁺X⁻ (R═CₙH₂ₙ₊₁, 0≦n≦16, but not limited thereto.

The “crosslinking region (LIII)” means a portion of the multi-functional group ligand comprising a functional group capable of crosslinking to an adjacent multi-functional group ligand, and preferably a core portion thereof. The “crosslinking” means that the multi-functional group ligand is bound to another adjacent multi-functional group ligand by intermolecular interaction. The intermolecular interaction includes a hydrophobic interaction, a hydrogen bond, a covalent bond (for example, a disulfide bond), a Van der Waals force, and an ionic bond, but not limited thereto. Therefore, the crosslinkable functional group can be variously selected according to the kind of the intermolecular interaction. The crosslinking region can comprise, for example, —SH, —NH₂, —COOH, -epoxy, -ethylene, -acetylene, -azide, —PO₃H, or —SO₃H, as a functional group.

TABLE 1
Exemplary functional groups of reactive region in multi-functional group ligand

| I | II | III |
|---|---|---|
| R—NH₂ | R′—COOH | R—NHCO—R′ |
| R—SH | R′—SH | R—SS—R′ |
| R—OH | R′-(Epoxy group) | R—OCH₂CH(OH)—R′ |
| R—NH₂ | R′-(Epoxy group) | R—NHCH₂CH(OH)—R′ |
| R—SH | R′-(Epoxy group) | R—SCH₂CH(OH)—R′ |
| R—NH₂ | R′—COH | R—N═CH—R′ |
| R—NH₂ | R′—NCO | R—NHCONH—R′ |
| R—NH₂ | R′—NCS | R—NHCSNH—R′ |
| R—SH | R′—COCH₃ | R′—COCH₂S—R |
| R—SH | R′—O(C═O)X | R—S(C═O)O—R′ |
| R-(Aziridine group) | R′—SH | R—CH₂CH(NH₂)CH₂S—R′ |
| R—CH═CH₂ | R′—SH | R—CH₂CH₂S—R′ |
| R—OH | R′—NCO | R′—NHCOO—R |
| R—SH | R′—COCH₂X | R—SCH₂CO—R′ |
| R—NH₂ | R′—CON₃ | R—NHCO—R′ |
| R—COOH | R′—COOH | R—(C═O)O(C═O)—R′ + H₂O |
| R—SH | R′—X | R—S—R′ |
| R—NH₂ | R′CH₂C(NH²⁺)OCH₃ | R—NHC(NH²⁺)CH₂—R′ |
| R—OP(O²⁻)OH | R′—NH₂ | R—OP(O²⁻)—NH—R′ |
| R—CONHNH₂ | R′—COH | R—CONHN═CH—R′ |
| R—NH₂ | R′—SH | R—NHCO(CH₂)₂SS—R′ |

(I: Functional group of reactive region in multi-functional group ligand, II: Active ingredient, and III: Exemplary bonds by reaction of I and II)

In the present invention, the compound which originally contains the above-described functional group can be used as a water soluble multi-functional group ligand, but a compound modified or prepared so as to have the above-described functional group by a chemical reaction known in the art can be also used as a water soluble multi-functional group ligand.

For the water soluble nanoparticles according to the present invention, one example of the multi-functional group ligand is dimercaptosuccinic acid, since dimercaptosuccinic acid originally contains the adhesive region, the crosslinking region, and the reactive region. That is, —COOH on one side of the dimercaptosuccinic acid functions to be bound to the nanoparticles with a disulfide bond and COOH and SH on the end portion function to bind to an active ingredient. As the functional group of the adhesive region (LI), —COOH can be used in addition to the dimercaptosuccinic acid, and as the functional group of the reactive region (LIII), a compound containing —COOH or —OH can be used as the multi-functional group ligand. Examples of the compound include dimercaptomaleic acid, and dimercaptopentadionic acid, but not limited thereto.

For the water soluble nanoparticles according to the present invention, another example of the preferable multi-functional group ligands is a protein. Protein is a polymer composed of more amino acids than peptides, that is, composed of several hundreds or several hundred thousands of amino acids, both terminals of which contain —COOH and a —NH₂ functional group, and several tens of —COOH, —NH₂, —SH, —OH, —CONH₂, and so forth. Since protein can naturally comprise an adhesive region, a crosslinking region, and a reactive region according to its structure, as the above-described peptide, it can be useful as a multi-functional group ligand of the present invention. Representative examples of proteins which are preferable as the phase transfer ligand include a structural protein, a storage protein, a transport protein, a hormone protein, a receptor protein, a contraction protein, a defense protein, and an enzyme protein. More specifically, albumin, an antibody, an antigen, avidin, streptavidin, protein A, protein G, protein S, immunoglobulin, lectin, selectin, angiopoietin, anticancer protein, antibiotic protein, hormone antagonist protein, interleukin, interferon, growth factor protein, tumor necrosis factor protein, endotoxin protein, lymphotoxin protein, a tissue plasminogen activator, urokinase, streptokinase, protease inhibitor, alkyl phosphocholine, surfactant, cardiovascular pharmaceutical protein, neuro pharmaceuticals protein and gastrointestinal pharmaceuticals.

For the water soluble nanoparticles according to the present invention, other examples of the preferable multi-functional group ligands include an amphiphilic ligand containing both of a hydrophobic region and a hydrophilic region. In the case of the nanoparticles synthesized in an organic solvent, hydrophobic ligands having long alkyl chain coat the surface. The hydrophobic region of the amphiphilic ligand, which was added at this time, and the hydrophobic ligand on the surface of the nanoparticles are bound to each other through intermolecular interaction to stabilize the nanoparticles. Further, the outermost part of the nanoparticles shows a hydrophilic functional group, and consequently water soluble nanoparticles can be prepared. Here, the intermolecular interaction includes a hydrophobic interaction, a hydrogen bond, and a Van der Waals force. Here, the portion which binds to the nanoparticles by the hydrophobic interaction is an adhesive region (LI), and further the crosslinking region (LII) and the reactive region (LIII) can be introduced therewith by an organochemical method. Further, in order to increase the stability in an aqueous solution, an amphiphilic polymer ligands with multiple hydrophobic regions and multiple hydrophilic regions can be used. Cross-linking between the amphiphilic ligands can also enhance colloidal stability of the nanoparticles in aqueous media. Hydrophobic region of the amphiphilic ligand can be a linear or branched structure composed of chains containing 2 or more carbon atoms, more preferably an alkyl functional group such as ethyl, n-propyl, isopropyl, n-butyl, isobutyl , t-butyl, octyl, decyl, tetradecyl, hexadecyl, icosyl, tetracosyl, dodecyl, cyclopentyl, and cyclohexyl; a functional group having an unsaturated carbon chain containing a carbon-carbon double bond, such as ethynyl, propenyl, isopropenyl, butenyl, isobutenyl, octenyl, decenyl and oleyl; and a functional group having an unsaturated carbon chain containing a carbon-carbon triple bond, such as propynyl, isopropynyl, butynyl, isobutynyl, octynyl and decynyl. Further, examples of the hydrophilic region include a functional group being neutral at a specific pH, or being positively or negatively charged at a higher or lower pH, such as —SH, —COOH, —NH₂, —OH, —PO₃H, —PO₄H₂, —SO₃H, —SO₄H, and —NR⁴⁺X⁻. Preferable examples thereof include a polymer and a block copolymer, wherein monomers used therefor include acrylic acid, alkylacrylic acid, ataconic acid, maleic acid, fumaric acid, acrylamidomethylpropanesulfonic acid, vinylsulfonic acid, vinylphosphoric acid, vinyllactic acid, styrenesulfonic acid, allylammonium, acrylonitrile, N-vinylpyrrolidone, and N-vinylformamide, but not limited thereto.

For the water soluble nanoparticles according to the present invention, another example of preferable multi-functional group ligands is a peptide. The peptide is an oligomer/polymer composed of several amino acids and since both ends of the amino acid contain —COOH and —NH₂ functional groups, peptide naturally comprises an adhesive region and a reactive region.

The multi-functional group ligand used in the present invention can be configured to be bonded to a biodegradable polymer. Examples of the biodegradable polymer include dextran, carbodextran, polysaccharide, cyclodextran, pullulan, cellulose, starch, glycogen, carbohydrate, monosaccharide, disaccharide, oligosaccharide, polyphosphazene, polylactide, polylactide-co-glycolide, polycaprolactone, polyanhydride, polymalic acid, a derivative of polymalic acid, polyalkylcyanoacrylate, polyhydroxybutyrate, polycarbonate, polyorthoester, polyethylene glycol, poly-L-lysine, polyglycolide, polymethylmethacrylate, and polyvinylpyrrolidone.

From another viewpoint, the present invention provides water soluble manganese-containing metal oxide hybrid nanoparticles, wherein a chemical molecule with biological function and a biomolecules are bonded to the reactive region of the water soluble manganese-containing metal oxide nanoparticles.

In the present invention, one example of the water soluble manganese-containing metal oxide hybrid nanoparticles is configured to have a chemical molecule bound to the water soluble manganese-containing metal oxide. Examples of the chemical molecule include various functional monomers, polymers, and inorganic supports. Examples of monomers include various kinds of the monomers including an anti-cancer agent, an antibiotic, a vitamin, a folic acid-containing drug, a fatty acid, a steroid, a hormone, purine, pyrimidine, a monosaccharide and a disaccharide, but not limited thereto. Examples of the polymer include dextran, carbodextran, polysaccharide, cyclodextran, pullulan, cellulose, starch, glycogen, carbohydrate, monosaccharide, disaccharide, oligosaccharide, polyphosphazene, polylactide, polylactide-co-glycolide, polycaprolactone, polyanhydride, polymalic acid and its derivatives, polyalkylcyanoacrylate, polyhydroxybutyrate, polycarbonate, polyorthoester, polyethylene glycol, poly-L-lysine, polyglycolide, polymethylmethacrylate, and polyvinylpyrrolidone. Examples of the inorganic support include silica (SiO₂), titania (TiO₂), indium tin oxide (ITO), carbon materials (nanotube, graphite, and fullerene), a semiconductor substrate (CdS, CdSe, CdTe, ZnO, ZnS, ZnSe, ZnTe, Si, GaAs, and AlAs), a metal substrate (Au, Pt, Ag, and Cu), but not limited thereto.

One example of the hybrid nanoparticles of the present invention is configured such that the water soluble manganese-containing metal oxide nanoparticles are selectively bound to the biomolecule. Examples of the biomolecule include tissue-specific binding substances such as protein, peptide, DNA, RNA, antigen, hapten, avidin, streptavidin, neutravidin, protein A, protein G, lectin, and selectin; pharmaceutical active ingredients such as an anti-cancer agent, an antibiotic, a hormone, a hormone antagonist, interleukin, interferon, a growth factor, a tumor necrosis factor, endotoxin, lymphotoxin, urokinase, streptokinase, a tissue plasminogen activator, a protease inhibitor, alkyl phosphocholine, a surfactant, cardiovascular pharmaceuticals, gastrointestinal pharmaceuticals, neuro pharmaceuticals; biologically active enzymes such as a hydrolase, a redox enzyme, a lyase, an isomerization enzyme, and a synthetase; an enzyme cofactor, and an enzyme inhibitor, but not limited thereto.

The water soluble manganese-containing metal oxide hybrid nanoparticles formed according to the present invention has an excellent magnetic moment as compared with the conventional MRI contrast agents comprising iron oxide, and thus it can allow a higher level of high-sensitivity diagnosis. Further, as compared with the conventionally used MRI contrast agent, even a small amount can provide an effect of enhancing the signals to a desired level. Accordingly, they can be used as a contrast agent having lower biological toxicity and side-effects than conventional materials.

Hereinbelow, the method of preparing the water soluble manganese-containing metal oxide nanoparticles of the present invention will be described in detail.

The water soluble manganese-containing metal oxide nanoparticles according to the present invention can be obtained by using a nanoparticles synthesis method in a gas phase or a nanoparticles synthesis method in a liquid phase including an aqueous solution, an organic solvent, or a multi-solvent system, which are known in the art.

As one example of the preferable methods of preparing the nanoparticles of the present invention, the nanoparticles can be prepared through the following steps: (1) synthesizing water-insoluble nanoparticles in an organic solvent, (2) dissolving the water-insoluble nanoparticles in a first solvent, and dissolving the water soluble multi-functional group ligands in a second solvent, and (3) mixing the two solutions obtained from the step (2) to conjugate with multi-functional group ligands on the surface of the water-insoluble nanoparticles followed by separation of by dissolving in an aqueous solution.

The step (1) of the method relates to a process for manufacturing water-insoluble nanoparticles. In one embodiment of the present invention, water-insoluble nanoparticles can be prepared by the method comprising the steps of introducing a nanoparticle precursor to an organic solvent containing a surface stabilizer at 10 to 600° C., maintaining a suitable temperature and period for preparing the desired water-insoluble nanoparticles, subjecting to chemical reaction to grow the nanoparticles, and then separating and purifying to prepare the resultant water-insoluble nanoparticles.

As the organic solvent, a benzene-based solvent (e.g., benzene, toluene, and halobenzene), a hydrocarbon solvent (e.g., octane, nonane, and decane), an ether-based solvent (e.g., benzyl ether, phenyl ether, and hydrocarbon ether), a polymer solvent, or an ionic liquid solvent can be used, but not limited thereto.

In the step (2) of the preparation method, the above-prepared nanoparticles are dissolved in the first solvent, while the multi-functional group ligand is dissolved in the second solvent. As the first solvent, a benzene-based solvent (e.g., benzene, toluene, and halobenzene), a hydrocarbon solvent (e.g., pentane, hexane, nonane, and decane), an ether-based solvent (e.g., benzyl ether, phenyl ether, and hydrocarbon ether), halo hydrocarbon (e.g., methylene chloride, and methane bromide), alcohols (e.g., methanol, and ethanol), a sulfoxide-based solvent (e.g., dimethylsulfoxide), an amide-based solvent (e.g., dimethylformamide), etc. can be used. As the second solvent, the solvent described above as the first solvent, as well as water can be used.

In the step (3) of the preparation method, the two solutions are mixed, such that the organic surface stabilizer of the water-insoluble nanoparticles is replaced with the water soluble multi-functional group ligand. The nanoparticles replaced with the water soluble multi-functional group ligand can be separated using a method known in the art. Generally, since the water soluble nanoparticles are generated as the precipitants, it is preferable that they are separated by centrifugation or filtration. After the separation, pH is preferably adjusted to 5 to 10 through a titration step to obtain water soluble nanoparticles which are more stably dispersed.

Further, in an alternative method, the water soluble nanoparticles of the present invention can be synthesized by crystal growth through a chemical reaction in an aqueous solution of a metal precursor. This method can be carried out by a known method for synthesizing water soluble nanoparticles, which is a method for synthesizing water soluble manganese-containing metal oxide nanoparticles by adding a manganese ion precursor in an aqueous solution comprising a multi-functional group ligand.

Hereinbelow, the application of the MRI contrast agent comprising water soluble manganese-containing metal oxide nanomaterials will be described in detail.

The water soluble manganese-containing metal oxide nanoparticles show much stronger amplification of spin-spin relaxation MRI signals (R2 relaxivity coefficient: about 360 L/mol/sec) than the conventional iron oxide nanoparticles. Accordingly, the water soluble manganese-containing metal oxide nanoparticles improve greatly the conventional diagnosis to allow early diagnosis of diseases and detection of traces of bio-molecules. Specific biological markers are generally over-expressed on the surface of the pathogens such as cancer cells. An antibody which can be selectively bound to such biological markers can be obtained by using a known method in the art. A previously known material can also be used. The materials (such as antibody) obtained by the method and the water soluble manganese-containing metal oxide nanoparticles are made to be bound to the reactive region according to the previously described method. As a result, the prepared hybrid nanoparticles can selectively bind to the cancer cells. The resulting magnetic particles which labels cancer cells allow the MRI signals to be visual, which makes the diagnosis possible.

Since the water soluble manganese-containing metal oxide nanoparticles have more excellent sensitivity, as compared with iron oxide nanoparticles which are conventionally used, it makes ultra-sensitive cancer diagnosis possible. Accordingly, the in vivo probing of small-sized cancers with the manganese-containing metal oxide nanoparticles makes it possible to diagnose cancer much earlier.

Further, the water soluble manganese-containing metal oxide nanoparticles in the present invention can release manganese ions in response to the external stimuli such as change in pH or temperatures. Since thus released manganese ions increase the T1 spin-lattice relaxation time in MRI, thus exhibiting a T1 contrast effect, it is possible to perform MRI diagnosis by release of manganese ions due to the environmental change in vivo.

The water soluble manganese-containing metal oxide nanoparticles can be also coupled to other diagnostic probes and used as a double- or multiple-diagnostic probe. For example, if a T1 MRI diagnostic probe is coupled to water soluble manganese-containing metal oxide, T2 MRI diagnosis and T1 MRI diagnosis can be simultaneously performed. Moreover, if coupled to an optical diagnostic probe, the magnetic resonance imaging and optical imaging can be simultaneously performed, and also, if coupled to a CT diagnostic contrast agent, the magnetic resonance imaging and the CT diagnosis can be simultaneously performed. In addition, if coupled to radioactive isotopes, the magnetic resonance imaging, and the PET, SPECT diagnosis can be simultaneously performed.

### MODE FOR THE INVENTION

Hereinbelow, the present invention will be described with reference to Examples only for an illustrative purpose. Thus, it will be apparent that Examples will not limit the scope of the present invention to a person with skill in the art to which this invention belongs to.

### EXAMPLES

### Example 1

### Comparison Between MRI Contrast Effects of Manganese Ferrite (MnFe₂O₄) Nanoparticles and those of Iron Oxide Nanoparticles, Cobalt Ferrite Nanoparticles, and Nickel Ferrite Nanoparticles

To confirm whether manganese ferrite nanoparticles (12 nm) as developed herein have an MRI contrast effect better than the conventional iron oxide nanoparticles and other metal ferrite nanoparticles, mass magnetization values, MR images and R2 spin-spin relaxation MRI of the iron oxide nanoparticles, cobalt ferrite nanoparticles and nickel ferrite nanoparticles (MFe₂O₄, M=Fe, Co, Ni) were measured.

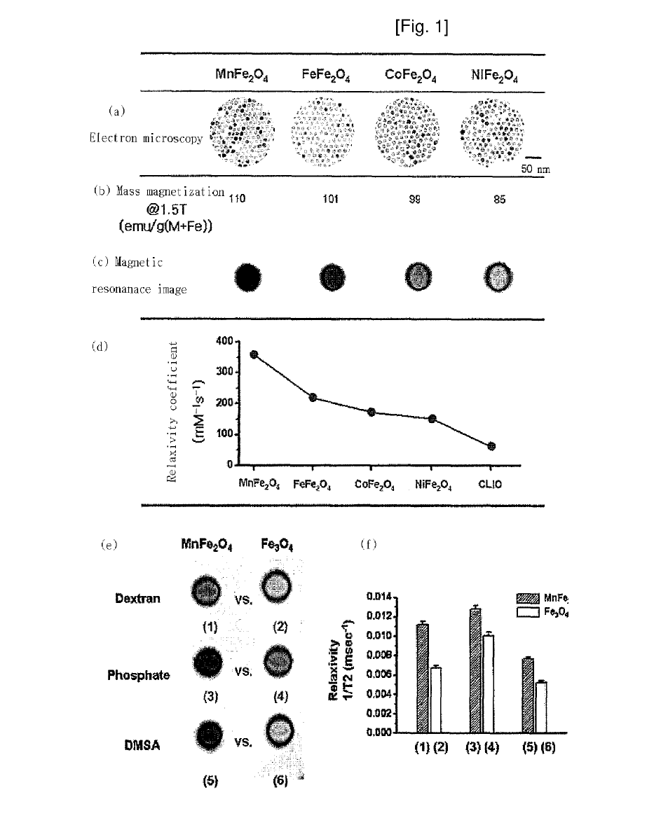

Above all, each nanoparticle was prepared in the same manners as disclosed in Korean Patent Nos. 10-0604976 and 10-0652251, PCT KR2004/002509, Korean Patent No. 10-0604976, PCT KR2004/003088, and Korean Patent Application No. 2006-0018921, and the obtained nanoparticles are sphere with a uniform size of 12 nm, as shown in FIG. 1(a), the surface thereof being coated with dimercaptosuccinic acid.

Magnetic susceptibility of each nanoparticle obtained, was measured using an MPMS superconducting quantum interference device (SQUID) magnetometer and observed with applying an external magnetic field varying in the range of −5 T to 5 T. As shown in FIG. 1(b), the manganese ferrite nanoparticles exhibited the highest magnetic property of 110 emu/g (Mn+Fe) (at 1.5 T), while iron oxide nanoparticles, cobalt ferrite nanoparticles, and nickel ferrite nanoparticles exhibited lower magnetic properties (101, 99, and 85 emu/g (M+Fe), respectively). Theses results are derived from the substitution effect of metal ion having each different d orbital spin moment in the metal ferrite nanoparticles having a spinel structure.

In order to demonstrate these MRI contrast effects of the nanoparticles, the T2-weighted magnetic resonance imaging was measured. For the measurement, 1.5 T system (Intera; Manufactured by Philips Medical Systems, Best, The Netherlands) equipped with micro-47 coils was used. The MR images were obtained using Carr-Purcell-Meiboom-Gill (CPMG) sequence. Specific parameters were as follows: point resolution of 156 μm×156 μm, section thickness of 0.6 mm, TE=20 ms, TR=400 ms, image excitation number of 1 and image acquisition time of 6 minutes. As shown in FIG. 1(c), it was found that manganese ferrite nanoparticles exhibited the strongest MRI signal (black color) and the MRI signals of iron oxide nanoparticles, cobalt ferrite nanoparticles, the nickel ferrite nanoparticles were decreased while changing gradually into light gray color. In the R2 relaxivity coefficient as a comparative measurement of a contrast effect, it was found that the coefficient of manganese ferrite nanoparticles is 358 mM⁻¹s⁻¹, which is an even more increased value, as compared with that of other metal ferrite nanoparticles having the same size and containing an iron oxide. The coefficient is five times increased value more than R2 coefficient of crosslinked iron oxide (CLIO) nanoparticles (68 mM⁻¹s⁻¹) which are hitherto known as the best MRI contrast agent in the art (FIG. 1(d)).

To confirm that these manganese ferrite nanoparticles exhibit the excellent MRI contrast effect, irrespective of the kinds of the coated ligands, the MRI contrast effects between manganese ferrite nanoparticles coated with various multifunctional group ligands and iron oxide nanoparticles were compared. As the ligand, in addition to dimercaptosuccinic acid suggested above, 3-carboxyl propylphosphonic acid and dextran which are generally used as ligands were used as examples.

As shown in FIG. 1(e, f), irrespective of the kinds of multifunctional group ligands, the manganese ferrite nanoparticles exhibited the increased MRI signal (black color) as compared with iron oxide nanoparticles. Further, as shown in the diagram of R2-relaxation time, it was found that the signal of water soluble manganese-containing metal oxide nanoparticles is 20 to 120% larger than that of conventional iron oxide nanoparticles.

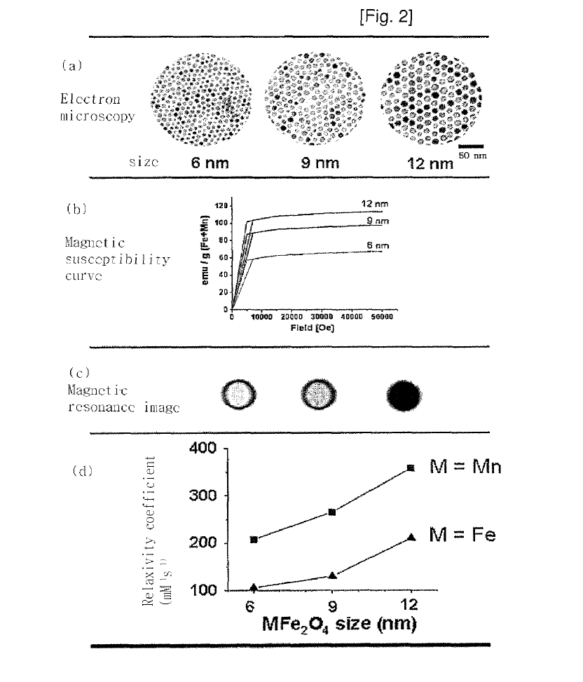

Since the size of particles significantly affects the MRI contrast effect, the contrast effects of manganese ferrite nanoparticles with various sizes were compared with those of iron oxide nanoparticles with the same size. To achieve this, the manganese ferrite nanoparticles and iron oxide nanoparticles with sizes of 6, 9 and 12 nm were prepared in the same manners as disclosed in Korean Patent No. 10-0604976, Korean Patent No. 10-0652251, PCT KR2004/002509, Korean Patent No. 10-0604976, PCT KR2004/003088, Korean Patent Application No. 2006-0018921. And magnetic resonance imaging was measured using the above mentioned Carr-Purcell-Meiboom-Gill (CPMG) sequence. TEM images of the prepared particles were shown in FIG. 2(a). It was found that a mass magnetization value of the obtained manganese ferrite nanoparticles increased as the sizes increased, as shown in FIG. 2(b). In accordance with this, it was found that as the sizes of manganese ferrite nanoparticles increased, MR imaging gradually changed to black and the signal increased (FIG. 2(c)), and it was found that the R2 relaxivity coefficient also increased (FIG. 2(d)). As compared with the contrast effects of iron oxide nanoparticles, it can be found that all manganese ferrite nanoparticles with sizes of 6, 9 and 12 nm have the increased contrast effects more than iron oxide nanoparticles.

### Example 2

### Evaluation on Colloidal Stability of Water Soluble Manganese Ferrite Nanoparticles Coated with Multifunctional Group Ligands in an Aqueous Solution

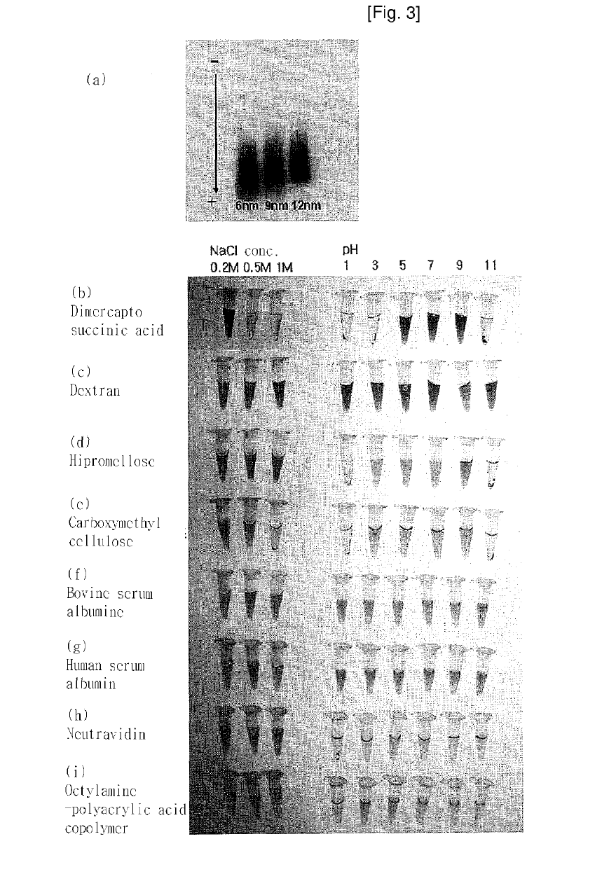

To evaluate the colloidal stability of the water soluble manganese ferrite nanoparticles in an aqueous solution, an agarose gel electrophoresis analysis and an investigation of the stability under the condition of various salt concentrations and acidities were carried out. Each manganese ferrite nanoparticle coated with various ligands was prepared in the same manners as disclosed in Korean Patent No. 10-0604976, Korean Patent No. 10-0652251, PCT KR2004/002509, Korean Patent No. 10-0604976, PCT KR2004/003088, and Korean Patent Application No. 2006-0018921. As shown in FIG. 2(a), it can be found that the nanoparticles coated with dimercaptosuccinic acid as a ligand moved to the (+) electrode, showing a thin band on agarose gel electrophoresis, whereby it can be confirmed that the nanoparticles are well dispersed with a uniform size without aggregation in an aqueous solution. Further, the stability of the water soluble manganese ferrite nanoparticles surface-stabilized with various water soluble ligands was evaluated (FIG. 3(b-i)), and as a result, it was confirmed that all kinds of the nanoparticles were stable in a salt concentration of 0.2 M and at pH 5 to 9, and the nanoparticles surface-stabilized with dextran, hipromellose, bovine serum albumin and human serum albumin, and neutravidin were stable even in a salt concentration of 1 M. Among these, the nanoparticles which were surface-stabilized by using dextran, bovine serum albumin and human serum albumin had very high colloidal stabilities in the wide range of acidities (pH 1 to pH 11). Further, the nanoparticles which were surface-stabilized with an octylamine-polyacrylic acid copolymer by a hydrophobic bond were stable in a salt concentration of 0.5 M and at pH 3 to 11. As considering that in vitro or in vivo test, the salt concentration was about 0.1 M, it is denoted that the nanoparticles have very high colloidal stabilities in an aqueous solution.

### Example 3

### Preparation for Manganese Ferrite Nanoparticles-Herceptin Hybrids for Diagnosis of Breast Cancer

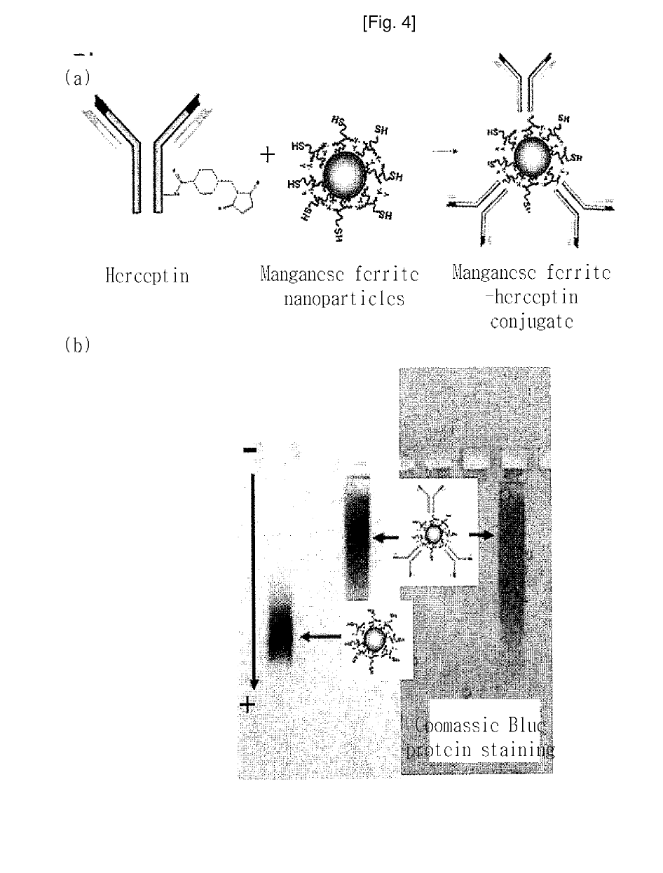

The diagram for summarizing the preparation process for nano hybrid material was shown in FIG. 4(a). 100 ml of herceptin [(10 mg/ml, in 10 mM sodium phosphate buffer, pH 7.2), manufactured by Genentech, Inc., South San Francisco, Calif., USA] was placed in an Eppendorf tube and 0.2 mg of sulfo-SMCC [40(N-maleimidomethyl)cyclohexane-1-carboxylic acid 3-sulfo-N-hydroxy-succimide ester] was added. The reaction was carried out at room temperature for 30 minutes to substitute the lysine residue of herceptin with a maleimide group. After an excessive amount of sulfo-SMCC molecules was removed through a Sephadex G-25 column, the maleimide-substituted herceptin was subjected to reaction with 200 ml of a solution containing water soluble manganese ferrite nanoparticles (10 mM PB, pH 7.2, 2 mg/ml) at room temperature for 24 hr. After completing the reaction, the mixture was passed through a Sephacryl S-300 column to remove the unreacted herceptin and the water soluble iron oxide nanoparticles. The resultant was concentrated to about 2 mg/ml using a centricon filtration kit to prepare manganese ferrite nanoparticles-herceptin hybrid. The prepared hybrid nanoparticles were analyzed by agarose gel electrophoresis. The result of Coomassie Blue protein staining confirmed that a nano hybrid material was prepared. (FIG. 4(b)).

### Example 4

### Identification of In Vitro Tumor Cell Selectivity of Manganese Ferrite Nanoparticle-Herceptin Hybrids and Comparison Thereof with Selectivity of Iron Oxide Hybrid Nanoparticles

In order to examine the binding specificity to and efficiency for HER2/neu antigen as a breast cancer marker antigen of the manganese ferrite nanoparticle-herceptin hybrids prepared in above Example 3, in vitro magnetic resonance imaging test was performed.

The process in which the manganese ferrite nanoparticles-herceptin hybrids were treated with each of the HER2/neu antigen nonexpressed, expressed and overexpressed cell lines was as follows. First, each cell line was harvested by treatment with 0.25% trypsin/EDTA at room temperature. The manganese ferrite nanoparticles-herceptin hybrids were added in a concentration of 2.5 nM in terms of the nanoparticles to 50 ml of a PBS buffer solution containing 10⁷ cells. The mixture was reacted at 4° C. for 30 minutes, and then washed three times. On the other hand, CLIO nonoparticle-herceptin hybrids were used as a control.

To examine the antigen specificity of the manganese ferrite nanoparticles-herceptin hybrids using magnetic resonance imaging, each cell line was transferred into a PCR tube and precipitated by centrifugation. The MRI contrast effect according to the antigen specificity of each cell line was evaluated by using a 1.5 T system (Intera; Manufactured by Philips Medical Systems, Best, The Netherlands) and micro-47 coils. Coronal images were obtained with fast field echo (FFE) pulse sequences. Specific parameters were as follows: point resolution of 156 □×156 □, section thickness of 0.6 mm, TE=20 ms, TR=400 ms, image excitation number of 1, and image acquisition time of 6 minutes. The MRI contrast effect according to the antigen specificity was quantitatively evaluated by using T2 mapping. Specific parameters were as follows: point resolution of 156 □×156 □, section thickness of 0.6 mm, TR=4000 ms, TE=20, 40, 60, 80, 100, 120, 140 and 160 ms, image excitation number of 2, and image ac quisition time of 4 minutes.

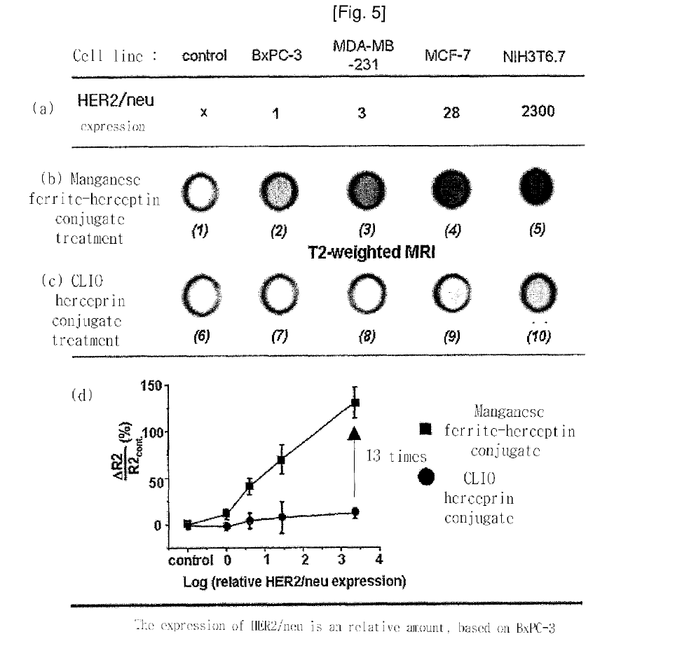

The results shown in FIG. 5 depict evaluation of the MR sensitivity of the manganese ferrite nanoparticles-herceptin hybrids for detection of the HER2/neu cancer markers. As known in FIG. 5, in the case of Bx-PC-3 cells (which have a relatively low HER2/neu expression level), the relative enhancement of the MRI contrast effect (ΔR2/Rcontrol) is ˜10% and the tumor markers were unambiguously detected (FIG. 5(a, b)). Further, in the case of MDA-MB-231, MCF-7, NIH3T6.7 cells, which express HER2/neu at higher levels, the relative enhancement of the MRI contrast effect (ΔR2/Rcontrol) is up to 40%, 70% and 130%, respectively (FIG. 5(a, b))

In contrast, when CLIO nonoparticle-herceptin hybrids were used as control, only NIH3T6.7 cells (which have a relatively high HER2/neu expression level) are detected with the relative enhancement of the MRI contrast effect of ˜10%. In cells which express HER2/neu at lower levels, the relative enhancement of the MRI contrast effect is 6% or less slightly (FIG. 5(c)).

As comparing the change between the R2 relaxivity coefficient in NIH3T6.7 cells treated with manganese ferrite nanoparticles-herceptin hybrids and that in NIH3T6.7 cells treated with CLIO nonoparticle-herceptin hybrids, it was found that in the case of using the manganese ferrite nanoparticles-herceptin hybrids in the invention, the R2 relaxivity coefficient was thirteen times higher. Further, as considering that Bx-PC-3 cells treated with the manganese ferrite nanoparticles-herceptin hybrids and NIH3T6.7 cells treated with the CLIO nonoparticle-herceptin hybrids exhibit the same enhancement of the MRI contrast effects and that the expression ratio of Bx-PC-3 cells to NIH3T6.7 cells is 1 to up to 2300, it was found that the manganese ferrite nanoparticles-herceptin hybrids have 2300 times higher detection limit for breast cancer markers than that of the conventional iron oxide nonoparticle-herceptin hybrids (FIG. 5(d)).

### Example 5

### Evaluation on Cell Stability of Manganese-Containing Metal Oxide

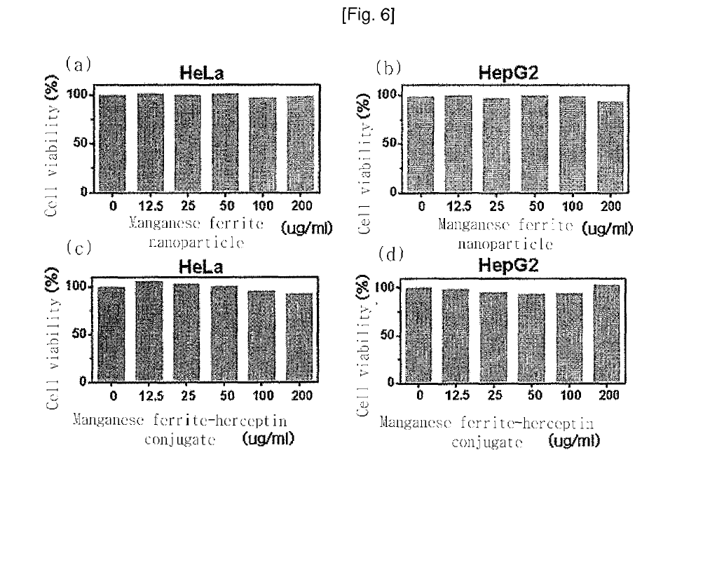

In order to use the particles as MRI contrast agents in vitro and in vivo, evaluation on the stability of the nanoparticles is also important. Accordingly, cytotoxicity tests of the dimercaptosuccinic acid-coated manganese ferrite nanoparticles prepared in Example 1 and of the manganese ferrite nanoparticles-herceptin hybrids used in Example 4 were performed. As shown in FIG. 6, it was found that both of the nanoparticles showed cell viabilities of almost up to 100% in the test concentration range up to 200 □/ml and did not show cytotoxicity.

### Example 6

### T2 MRI Diagnosis Using Manganese-Containing Metal Oxide (MN₃O₄)

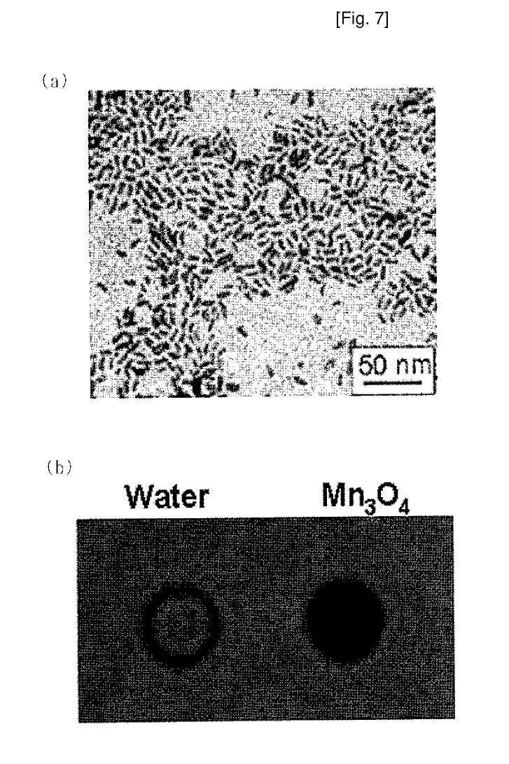

To evaluate the T2 MRI effect of the water soluble manganese-containing metal oxide nanoparticles, T2 mapping for a solution containing the manganese-containing metal oxide nanoparticles with a particle size of 3 nm×8 nm was performed. As shown in FIG. 6, the manganese-containing metal oxide nanoparticles were prepared in the same manners as disclosed in Korean Patent No. 10-0604976, Korean Patent No. 10-0652251, PCT KR2004/002509, Korean Patent No. 10-0604976, PCT KR2004/003088, and Korean Patent Application No. 2006-0018921. The electron microscopic pictures of the prepared particles were shown in FIG. 7(a).

It was found that a manganese-containing metal oxide nanoparticles-containing solution had significant contrast effects in T2 MRI than a solution not containing manganese-containing metal oxide nanoparticles (FIG. 7). Therefore, the manganese-containing metal oxide nanoparticles can be used as T2 contrast agent.

### Example 7

### T1 MRI Diagnosis Using the Releasing Effect of Manganese Ion

To confirm whether a T1 MRI diagnosis is available or not by using a releasing effect of ion in manganese ferrite and manganese-containing metal oxide nanoparticles, a T1 MRI was measured with pH variation. The MRI contrast effect was evaluated by using 1.5 T system (Intera; Manufactured by Philips Medical Systems, Best, The Netherlands) and micro-47 coils. Coronal images were obtained with fast field echo (FFE) pulse sequences. Specific parameters were as follows: point resolution of 156 □×156 □, section thickness of 0.6 mm, TE=20 ms, TR=400 ms, image excitation number of 1, and image acquisition time of 6 minutes. Manganese ferrite nanoparticles with the particle size of 12 nm prepared in Example 1 and manganese-containing metal oxide nanoparticles prepared in Example 6 were used.

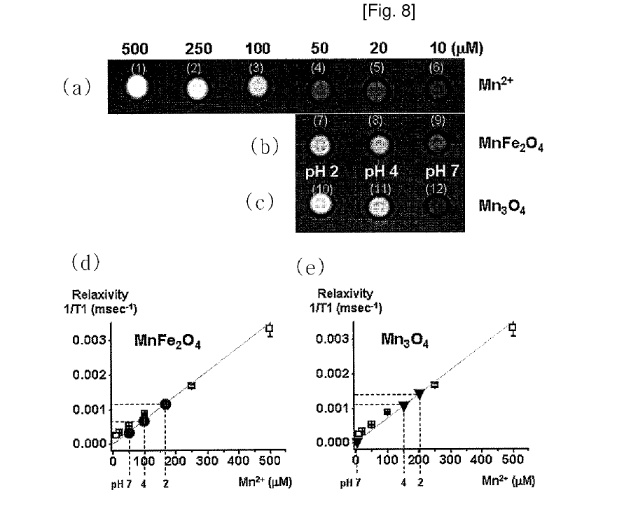

As shown in FIG. 8(b), in a neutral solution, the manganese ferrite nanoparticles exhibit very weak T1 contrast effect (FIG. 8b(9)). However, in acidic solutions (pH=2, 4), the manganese ferrite nanoparticles exhibit the contrast effect that T1 signals change to bright colors due to the release of Mn²⁺ in the MR images (FIG. 8b(7, 8)). Further, as shown in FIG. 8(c), in neutral solutions, the manganese-containing metal oxide nanoparticles never exhibit T1 contrast effect (FIG. 8c(12)). However, in acidic solutions (pH=2), the manganese-containing metal oxide nanoparticles exhibit the contrast effect that T1 signals change to bright colors due to the release of Mn²⁺ in MR images (FIG. 8c(11, 16)).

For quantitative evaluation for Mn²⁺ from the nanoparticles dissolved, T1 of the solutions containing Mn²⁺ ions in a determined concentration was measured to plot calibration curves (FIG. 8(a)). As shown in FIG. 8(e), from the calibration curves, it was found that in the solution containing the manganese-containing metal oxide nanoparticles, 150 μM of Mn²⁺ ions exist at pH 4 and 200 μM of Mn²⁺ ion exist at pH 2. As such, from the calibration curves, it was also found that in the solution containing the MnFe₂O₄ nanoparticles, the concentration of the Mn²⁺ ions increased at pH 2 and 4 (FIG. 8(d)).

Accordingly, it was found that the manganese-containing metal oxide nanoparticles can be used as a diagnostic probe which exhibits a T1 contrast effect by injecting the manganese-containing metal oxide nanoparticles into a specific region and releasing the Mn²⁺ in response to external stimulus.

### Example 8

### In Vivo Tumor Diagnosis with High Sensitivity Using Water Soluble Manganese Ferrite Nanoparticle-Herceptin Conjugate Nanosystem

A small size of a breast cancer tissue was diagnosed successfully on in vivo MRI using a water soluble manganese ferrite-herceptin hybrid nanosystem. The manganese ferrite nanoparticles nonoparticle-herceptin hybrids was prepared in the same manners as in Example 3. A set of nude mice subjects were implanted with NIH3T6.7 cell lines in which Her2/neu markers were overexpressed. After three days, the nude mice (n=8) having a tumor size of 5 mm×5 mm×2 mm were injected via tail vein with the hybrid in a concentration of 20 mg/kg. In the durations of 1, 2 and 8 hours after injection, MR imaging of the mice was performed. In parallel, the same experiment was performed using CLIO nonoparticle-herceptin hybrids and iron oxide (Fe₃O₄) nonoparticle-herceptin hybrids as control.

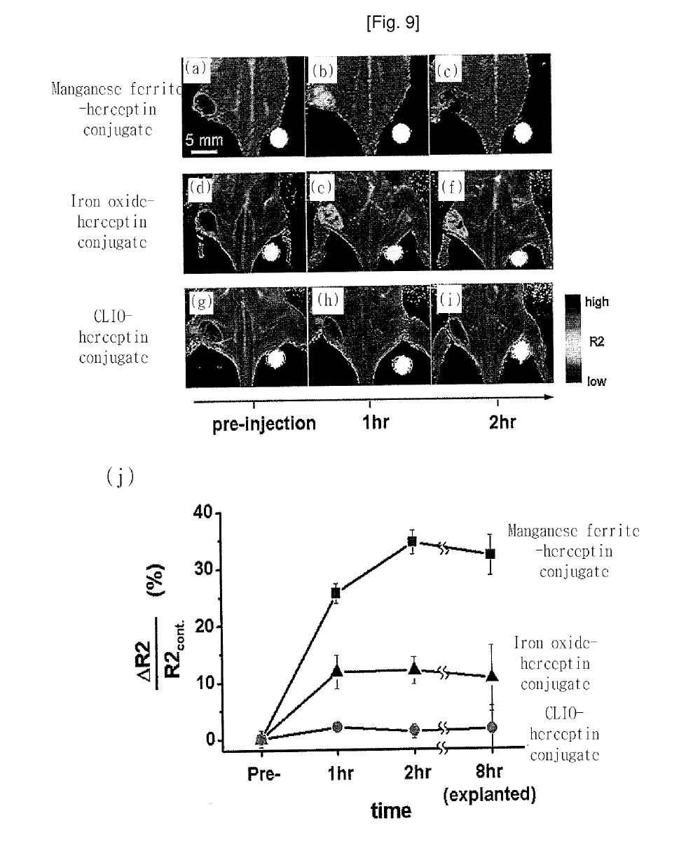

As results of the color mapped MRI shown in FIG. 9, there can be seen that MR image (FIG. 9(a-c)) of the tumor site in the mice treated with the manganese ferrite nanoparticles-herceptin hybrids completely change from red to blue at the temporal points of 2 hours, relative to that at preinjection, as compared with the iron oxide nonoparticle-herceptin hybrids (FIG. 9(d to f)) and the CLIO nonoparticle-herceptin hybrids (FIG. 9(h to i)). On the other hand, the MR image of the tumor site in the mice treated with the iron oxide nonoparticle-herceptin hybrids changes from red to mixed color (red and yellow) at the temporal points of 2 hours, and the MR image of the tumor site in the mice with the CLIO nonoparticle-herceptin hybrids never change at the temporal points of 2 hours. Further, as the MR R2 variance (ΔR2/R2control) in tumor sites to each nanoparticle shown in FIG. 9(j), while 35% change of the R2 relaxivity coefficient was observed in the mice treated with the manganese ferrite nanoparticles-herceptin hybrids at the temporal points of 8 hours, 10% and 3% change of the R2 relaxivity coefficient were observed in the mice treated with the iron oxide nonoparticle-herceptin hybrids and the CLIO nonoparticle-herceptin hybrids, respectively.

Therefore, if the manganese ferrite nanoparticles-herceptin hybrids are used as an MRI contrast agent for cancer diagnosis, they will lead to more excellent enhancement of the MRI contrast effect, as compared with the conventional nanoparticles such as —iron oxide and CLIO—. And the diagnosis of the small size of tumors was achieved.

### Example 9

### In Vivo Distribution of Manganese Ferrite Nanoparticles-Herceptin Hybrids Labeled with Radioactive Isotope ¹¹¹In

In vivo distribution of manganese ferrite nanoparticle-herceptin hybrids was analyzed by labeling with radioactive isotope ¹¹¹In. The mouse for vivo test is a mouse (n=3) having the same condition as in Example 7. The manganese ferrite nanoparticles-herceptin hybrids labeled with radioactive isotope ¹¹¹In were prepared as follows. First, 10 mg of herceptin was dissolved in 1 ml of 2.5 mM sodium acetate buffer (pH 6.5), and then mixed with 1 mg of DTPA (diethylene triamine pentaacetate) and 1 mg of sulfo-SMCC. After 1 hour, the maleimide/DTPA-activated herceptin was purified by applying the mixture to a Sephadex G-25 column, and immediately mixed with 4 mg of water soluble manganese ferrite nanoparticles to carry out the reaction. After 4 hours, the reaction mixture was then passed through a Sephacryl S-300 column to remove unreacted herceptin and nanoparticles, and then 3 mCi of ¹¹¹InCl₃ was added to the solution to carry out the reaction. After 1 hour, the manganese ferrite nanoparticles-herceptin hybrids labeled with ¹¹¹In were purified by applying the mixture to a Sephadex G-25 column, and then 0.4 mg (M+Fe) of the solution injected to mice via tail vein. An analysis of in vivo distribution using g-camera and g-counter was followed.

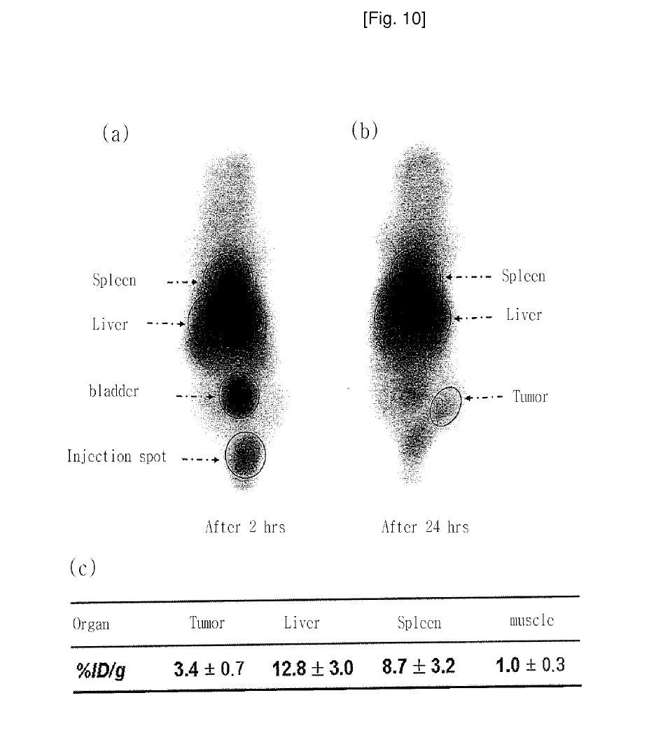

As shown in FIG. 10(a, b), after 2 hours, the hybrids were distributed in the liver, spleen, bladder or the like, and the strong signal was observed at the injected region of tail. However, after 24 hours, the signal became weak at the injected region of tail and detected the signal at tumor site. And then each organ was harvested, in vivo distribution using g-counter was analyzed. As shown in FIG. 10(c), signal of 12.8±3.0, 8.7±3.2 and 1.0±0.3% ID/g were observed in liver, spleen and muscle, in vivo distribution of 3.4±0.7% ID/g was observed in tumor.

### Example 11

### Optical-MRI Dual Mode Diagnostic Hybrid Nanosystem

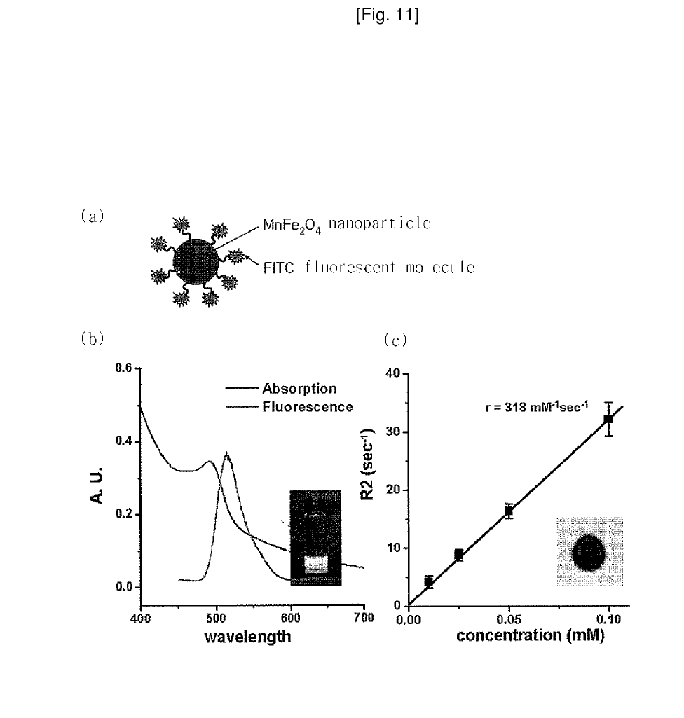

To develop a diagnostic probe simultaneously having optical and magnetic properties, the manganese ferrite nanoparticles surface-stabilized with bovine serum albumin were labeled with fluorochrome (FITC) to develop conjugate particles having both of the magnetic properties and the fluorescence (FIG. 11 a). About 20-fold excessive amount of NHS-FITC was added, based on —NH₂ molar ratio in bovine serum albumin, and the mixture was subject to reaction in 10 mM of phosphate buffered saline for 2 hours at ambient temperature. The excessive amount of unreacted NHS-FITC was removed by dialysis (MWCO, ˜2000) in the buffer solution. As shown in FIG. 11, it was found that the present optical-magnetic conjugate particles have both of the fluorescence and the MRI signals.

1. An MRI contrast agent comprising water soluble manganese-containing metal oxide nanoparticles.

2. The MRI contrast agent according to claim 1, wherein the water soluble manganese-containing metal oxide nanoparticles are obtained by the chemical reaction of a manganese precursor either in a gas phase, or in a liquid phase selected from the group consisting of an aqueous solution, an organic solvent, and a multi-solvent system.

3. The MRI contrast agent according to claim 1, wherein the water soluble manganese-containing metal oxide nanoparticles has a solubility in water of at least 1 □/ml and a hydrodynamic radius of the nanoparticle dissolved in water of 1000 nm or less.

4. The MRI contrast agent according to claim 1, wherein the water soluble manganese-containing metal oxide nanoparticles have their core consisting of 1 to 1000 nm-sized manganese-containing metal oxide nanoparticles, and comprise MnOₐ (0<a≦5) or MnM_{b}O_{c}, wherein M is at least one metal atoms selected from the group consisting of a Group 1 or 2 element such as Li, Na, Be, Ca, Ge, Mg, Ba, Sr and Ra, a Group 13 element such as Ga and In, a transition metal element such as Y, Ta, V, Cr, Co, Fe, Ni, Cu, Zn, Ag, Cd and Hg, and lanthanide or actinide group elements such as La, Ce, Pr, Nd, Pm, Sm, Eu, Gd, Tb, Dy, Ho, Er, Tm and Yb, wherein 0<b≦5, and 0<c≦10.

5. The MRI contrast agent according to claim 1, wherein the water soluble manganese-containing metal oxide nanoparticles comprise MnM′_{d}FeₑO_{f}, wherein M′ is at least one metal atom selected from the group consisting of a Group 1 or 2 element such as Li, Na, Be, Ca, Ge, Mg, Ba, Sr and Ra, a Group 13 element such as Ga and In, a transition metal element such as Y, Ta, V, Cr, Co, Fe, Ni, Cu, Zn, Ag, Cd and Hg, and lanthanide or actinide group elements such as La, Ce, Pr, Nd, Pm, Sm, Eu, Gd, Tb, Dy, Ho, Er, Tm and Yb, wherein 0<d≦5, 0<e≦5, and 0<f≦15.

6. The MRI contrast agent according to claim 1, wherein the water soluble manganese-containing metal oxide nanoparticle is at least one selected from the group consisting of Mn_{g}FeₕO₄ (0<g≦4, 0<h≦4), MnᵢFeⱼZnₖO₄ (0<i≦4, 0<j≦4, 0<k≦4) and MnₓFe_{y}Cu_{z}O₄ (0<x≦4, 0<y≦4, 0<z≦4).

7. The MRI contrast agent according to claim 1, wherein the water soluble manganese-containing metal oxide nanoparticle is at least one selected from the group consisting of MnO, Mn₂O₃, MnO₂, Mn₃O₄, and Mn₂O₅.

8. The MRI contrast agent according to claim 1, wherein the water soluble manganese-containing metal oxide nanoparticles are soluble in water themselves, or coated with a water soluble multi-functional group ligand.

9. The MRI contrast agent according to claim 8, further comprises a water soluble multi-functional group ligand which is attached to a surface of water soluble manganese-containing metal oxide nanoparticles via any one bond of an ionic bond, a covalent bond, a hydrogen bond, a hydrophobic bond, and a metal-ligand coordination bond.

10. The MRI contrast agent according to claim 9, wherein the water soluble multi-functional group ligand comprises an adhesive region (LI) for binding to the water soluble manganese-containing metal oxide nanoparticles.

11. The MRI contrast agent according to claim 10, wherein the water soluble multi-functional group ligand further comprises:
a reactive region (LII) for binding to an active ingredient;
a crosslinking region (LIII) for crosslinking between the ligands; or
a reactive region (LII)-crosslinking region (LIII) which includes both the reactive region (LII) and the crosslinking region (LIII).

12. The MRI contrast agent according to claim 10, wherein the adhesive region (LI) comprises a functional group selected from the group consisting of —COOH, —NH₂, —SH, —CONH₂, —PO₃H, —PO₄H, —SO₃H, —SO₄H, —OH, and hydrocarbon having two or more carbon atoms.

13. The MRI contrast agent according to claim 11, wherein the reactive region (LII) comprises at least one functional group selected from the group consisting of —SH, —COOH, —NH₂, —OH, —NR₃⁺X⁻, —N₃, —SCOCH₃, —SCN, an epoxy group, a sulfonate group, a nitrate group, a phosphonate group, an aldehyde group, a hydrazone group, alkene and alkyne.

14. The MRI contrast agent according to claim 11, wherein the water soluble multi-functional group ligand is a peptide comprising at least one amino acid having —SH, —COOH, —NH₂ and —OH as a side chain.

15. The MRI contrast agent according to claim 11, wherein the water soluble multi-functional group ligand comprises a —COOH group as a functional group of the adhesive region (LI), and a —COOH group or a —SH group as a functional group of the reactive region (LII).

16. The MRI contrast agent according to claim 11, wherein the water soluble multi-functional group ligand comprises a hydrocarbon chain having two or more carbon atoms as a functional group of the adhesive region (LI), and —COOH, —SH, —NH₂, —POₓH (0<x≦4), —SO_{y}H (0<x≦4), —NR₄⁺X⁻ (R═CₙHₘ 0≦n≦16, 0≦m≦34, X═OH, Cl, or Br) or —OH as a functional group of the reactive region (LII).

17. The MRI contrast agent according to claim 9, wherein the water soluble multi-functional group ligand is at least one selected from the group consisting of dimercaptosuccinic acid, dimercaptomaleic acid and dimercaptopentadionic acid.

18. The MRI contrast agent according to claim 9, wherein the water soluble multi-functional group ligand comprises at least one selected from the group consisting of dextran, carbodextran, polysaccharide, cellulose, starch, glycogen, carbohydrate, monosaccharide, disaccharide, oligosaccharide, polyphosphazene, polylactide, polylactide-co-glycolide, polycaprolactone, polyanhydride, polymalic acid, a derivative of polymalic acid, polyalkylcyanoacrylate, polyhydroxybutyrate, polycarbonate, polyorthoester, polyethylene glycol, poly-L-lysine, polyglycolide, polymethylmethacrylate, and polyvinylpyrrolidone.

19. The MRI contrast agent according to claim 9, wherein the water soluble multi-functional group ligand is at least one selected from the group consisting of peptides, albumins, avidins, antibodies, secondary antibodies, cytochrome, casein, myosin, glycinin, carotene, collagen, global proteins, and light proteins.

20. An MRI contrast agent comprising water soluble manganese-containing metal oxide hybrid nanoparticles which are configured to have an active ingredient bound to a reactive region (LII) of the water soluble multi-functional group ligand.

21. The MRI contrast agent according to claim 20, wherein the active ingredient is selected from a chemically functional monomer, a polymer, an inorganic support, and a biologically functional material.

22. The MRI contrast agent according to claim 21, wherein the chemically functional monomer is at least one selected from the group consisting of an anti-cancer agent, an antibiotic, a vitamin, a folic acid containing drug, a fatty acid, a steroid, a hormone, purine, pyrimidine, a monosaccharide and a disaccharide.

23. The MRI contrast agent according to claim 21, wherein the polymer is at least one selected from the group consisting of dextran, carbodextran, polysaccharide, cyclodextran, pullulan, cellulose, starch, glycogen, carbohydrate, oligosaccharide, polyphosphazene, polylactide, polylactide-co-glycolide, polycaprolactone, polyanhydride, polymalic acid and a derivative of polymalic acid, polyalkylcyanoacrylate, polyhydroxybutyrate, polycarbonate, polyorthoester, polyethylene glycol, poly-L-lysine, polyglycolide, polymethylmethacrylate, and polyvinylpyrrolidone.

24. The MRI contrast agent according to claim 21, wherein the inorganic support is at least one selected from the group consisting of silica (SiO₂), titania (TiO₂), ITO (indium tin oxide), zirconia (ZrO₂), and a semiconductor comprising gallium Arsenide (GaAs), silicon (Si), zinc oxide (ZnO), zinc sulfate (ZnS), zinc selenide (ZnSe), zinc telluride (ZnTe), cadmium sulfate (CdS), cadmium selenide (CdSe), cadmium telluride (CdTe), lead sulfate (PbS), lead selenide (PbSe), and lead telluride (PbTe).

25. The MRI contrast agent according to claim 21, wherein the biologically functional material is at least one selected from the group consisting of nucleic acids such as DNA and RNA, peptides, antigens, antibodies, haptens, avidins, neutravidin, streptavidin, protein A, protein G, lectin, selectin, an anti-cancer agent, an antibiotic, a hormone, a hormone antagonist, interleukin, interferon, a growth factor, a tumor necrosis factor, endotoxin, lymphotoxin, urokinase, streptokinase, a tissue plasminogen activator, a protease inhibitor, alkyl phosphocholine, a surfactant, an aptamer, a protein drug, biologically active enzymes such as a hydrolase, a redox enzyme, a lyase, an isomerization enzyme, and a synthetase; an enzyme cofactor, and an enzyme inhibitor.

26. The MRI contrast agent according to claim 1, said MRI contrast agent being used for T2 spin-spin relaxation MRI sequence.

27. The MRI contrast agent according to claim 1, said MRI contrast agent being used for T1 spin-lattice relaxation MRI detecting the release of Mn²⁺ caused by an external stimuli or the environmental change in vivo.

28. The MRI contrast agent according to claim 1, wherein the water soluble manganese-containing metal oxide nanoparticles comprise a radioactive isotope material.

29. The MRI contrast agent according to claim 28, said MRI contrast agent being used for Single Positron Emission Computer Tomography (SPECT) or Positron Emission Tomography (PET).

30. The MRI contrast agent according to claim 1, wherein the water soluble manganese-containing metal oxide nanoparticles comprise a fluorescent material.

31. The MRI contrast agent according to claim 30, said MRI contrast agent being used for the optical imaging and spectroscopy.
